(19) United States
(12) Patent Application Publication
(10) Pub. No.: US 2016/0331237 A1
(43) Pub. Date: Nov. 17, 2016

(54) Acoustic Detection of Bone Fracture

(71) Applicant: The Seaberg Company, Inc., Wilsonville, OR (US)

(72) Inventors: Lance David Hopman, Tigard, OR (US); Samuel Scheinberg, Portland, OR (US); William Henry Fox, III, Portland, OR (US)

(73) Assignee: The Seaberg Company, Inc., Wilsonville, OR (US)

(21) Appl. No.: 14/710,401

(22) Filed: May 12, 2015

(51) Int. Cl. A61B 5/00

(52) U.S. Cl. CPC A61B 5/0051; A61B 5/4504; A61B 5/0002

(57) ABSTRACT

A system for analyzing a possibility of a stress fracture in a patient's bone by applying a vibration of a selected frequency to a patient at a selected anatomical location and analyzing the resulting vibration detected at another anatomical location. Analysis may be based on a database. A probability of the existence of a fracture may be displayed. System software may provide usage instructions.

### BACKGROUND OF THE INVENTION

Physicians typically diagnose fractures by physically examining the patient and/or performing X-ray radiography. In some circumstances an X-ray will not show a fracture. This is especially common with some wrist fractures, hip fractures (especially in older people), and stress fractures. In these situations, the physician may perform other costly tests, such as a computed tomography (CT) scan, magnetic resonance imaging (MRI), or a bone scan (radionuclide scintigraphy).

Undiagnosed fractures are potentially dangerous. For example, undiagnosed stress fractures may develop into an acute fracture if left untreated. An injured person such as an athlete may not know the cause of experienced pain and so may aggravate the stress fracture through continued activity. Likewise, any elderly person sustaining a ground level fall has a risk of hip fracture, which oftentimes goes undiagnosed.

Use of smart phones in medical diagnoses and use of bone conduction of sound in diagnosis of bone fractures has been noted in literature, including the following:

Chandrasekaran V, Dantu R, Jonnada S, Thiyagaraja S, Subbu K P, Cuffless differential blood pressure estimation using smart phones. IEEE Trans Biomed Eng. 2013 April; 60(4):1080-9. Smart phones today have become increasingly popular with the general public for their diverse functionalities such as navigation, social networking, and multimedia facilities. These phones are equipped with high-end processors, high-resolution cameras, and built-in sensors such as accelerometer, orientation-sensor, and light-sensor. According to comScore survey, 26.2% of U.S. adults use smart phones in their daily lives. Motivated by this statistic and the diverse capability of smart phones, we focus on utilizing them for biomedical applications. We present a new application of the smart phone with its built-in camera and microphone replacing the traditional stethoscope and cuff-based measurement technique, to quantify vital signs such as heart rate and blood pressure. We propose two differential blood pressure estimating techniques using the heartbeat and pulse data. The first method uses two smart phones whereas the second method replaces one of the phones with a customized external microphone. We estimate the systolic and diastolic pressure in the two techniques by computing the pulse pressure and the stroke volume from the data recorded. By comparing the estimated blood pressure values with those measured using a commercial blood pressure meter, we obtained encouraging results of 95-100% accuracy.

Matzek B A, Fivecoat P T, Ritz R B. Novel approach to the diagnosis of fractures in an austere environment using a stethoscope and a cellular phone. Wilderness Environ Med. 2014 March; 25(1):99-102. a. BACKGROUND: Fracture diagnosis in the austere environment where radiographic tests are not available can be a challenge. In the past, a diagnostic technique has been described using a tuning fork and stethoscope to assess decreased sound conduction in the fractured extremity. In this study, we evaluate the use of a cellular phone's vibrate function and a stethoscope to limit equipment carried by expeditionary practitioners. OBJECTIVE: The purpose of this study was to evaluate the accuracy of fracture diagnosis using a cellular phone and stethoscope. METHODS: This is a pilot study to assess the usefulness of the above technique before clinical implementation. In 3 cadavers, we created fractures of the humerus and femur. Twenty-seven emergency medicine residents and an attending physician performed the diagnostic technique. RESULTS: Overall, the use of the cellular phone and stethoscope resulted in a sensitivity of 73% (95% confidence interval [CI]: 0.64 to 0.81) and a specificity of 83% (95% CI: 0.77 to 0.88), with a positive predicted value of 68% (95% CI: 0.59 to 0.77) and a negative predicted value of 86% (95% CI: 0.81 to 0.90). Positive likelihood ratio was 4.3, and negative likelihood ratio was 0.32. CONCLUSIONS: The use of a cellular phone and stethoscope may be a useful tool for the diagnosis of fractures in the austere environment. However, further study is needed to validate these findings in the clinical environment.

Moore M B. The use of a tuning fork and stethoscope to identify fractures. J Athl Train. 2009 May-June; 44(3):272-4. a. CONTEXT: Nonradiographic tests to identify fractures rely on a patient's report of increased pain at the site of injury. These tests can be misleading and produce false-positive or false-negative results because of differences in pain tolerance. A painless technique using a tuning fork and stethoscope to detect fractures has undergone limited review in the athletic training literature. OBJECTIVE: To determine if the use of a 128-Hz vibrating tuning fork and stethoscope were effective in identifying fractures. DESIGN: Cross-sectional study. SETTING: University athletic training room or local orthopaedic center when fractures were suspected. PATIENTS OR OTHER PARTICIPANTS: A total of 37 patients (19 males, 18 females) volunteered. MAIN OUTCOME MEASURE(S): A diminished or absent sound arising from the injured bone as compared with the uninjured bone represented a positive sign for a fracture. Radiographs interpreted by the attending orthopaedic physician provided the standard for comparison of diagnostic findings. RESULTS: Sensitivity was 0.83 (10:12), specificity was 0.80 (20:25), positive likelihood ratio was 4.2, negative likelihood ratio was 0.21, and diagnostic accuracy was 81% (30:37). CONCLUSIONS: The tuning fork and stethoscope technique was an effective screening method for a variety of fractures.

Misurya R K, Khare A, Mallick A, Sural A, Vishwakarma G K. Use of tuning fork in diagnostic auscultation of fractures. Injury. 1987 January; 18(1):63-4. a. This study was conducted on 50 patients in the Central Institute of Orthopaedics, Safdarjung Hospital, New Delhi, from June to October 1985. With the help of a child's stethoscope and a tuning fork of 128 Hz, the sound conducted by an injured limb was compared with that by the uninjured limb. The presence of a fracture reduced or abolished the conduction of sound by a bone. This method allows a quick examination without causing any pain, which is an advantage in an uncooperative patient. It is also reliable in the unconscious. The test is so simple that paramedical staff can use it. The results were correct in 94 percent of patients and were confirmed by radiological examination whereas clinical diagnosis was correct in only 88 percent of cases.

Borgerding L J, Kikillus P J, Boissonnault W G. Use of the patellar-pubic percussion test in the diagnosis and management of a patient with a non-displaced hip fracture. J Man Manip Ther. 2007; 15(4):E78-84. a. This case report describes the diagnosis and subsequent medical and physical therapy management of a 68-year-old patient with an undiagnosed non-displaced hip fracture. Initial plain film radiographs and a computed tomography (CT) scan of the involved hip were both interpreted as negative. One of the findings on the physical examination included a positive patellar-pubic percussion test (PPPT). This finding in a female patient of this age raised the suspicion of an occult hip fracture and she was referred back to her primary care physician. Repeat radiographs revealed a non-displaced hip fracture and the patient was treated surgically. The PPPT is an easy-to-implement clinical examination tool that may be extremely useful in physical therapy practice to guide the decision-making process for patients with suspected hip fractures. The utilization of the PPPT by the treating physical therapist for the patient in this case report contributed to a timely diagnosis, potentially preventing the disabling sequalae associated with a displaced femoral fracture.

Adams S L, Yarnold P R. Clinical use of the patellar-pubic percussion sign in hip trauma. Am J Emerg Med. 1997 March; 15(2):173-5. a. To assess the reliability and validity of osteophony (patellar-pubic percussion [PPP] test) as a physical diagnostic sign in the evaluation of hip trauma, a prospective study was undertaken of 41 consecutive patients presenting to the emergency department with a history of hip trauma necessitating radiographic examination. Fifteen of 19 (78.9%) patients who presented with a history of hip trauma and a fracture on radiograph were found to have had an abnormal PPP sign by at least one of two raters (P<0.0001). Only 1 of 22 (4.6%) patients without evidence of fracture (e.g., contusion) had an abnormal PPP sign. This patient had diffuse Paget's disease. Nine of 10 (90%) patients who had trochanteric fractures had an abnormal PPP sign (P<0.02). Overall reliability of the PPP sign based on two observers was 90.2% (P<0.0001). In those patients with radiographic evidence of fracture, interrater reliability was 84.2% (P<0.0001). For patients in whom physicians agreed on the PPP sign, the PPP test resulted in a 0% false-positive error and a 25% false-negative error. For patients in whom either physician noted an abnormal PPP sign, the PPP test resulted in a 4.6% false-positive error and a 21.1% false-negative error. The presence of an abnormal PPP sign in the evaluation of hip trauma is associated with evidence of fracture or other bony abnormality on radiograph.

Siffert R S, Kaufman J J. Acoustic assessment of fracture healing. Capabilities and limitations of “a lost art”. Am J Orthop (Belle Mead N.J.). 1996 September; 25(9):614-8. a. The ability of bone to conduct sound was applied clinically over 50 years ago to identify the presence of fresh fractures, although the technique has become a relatively “a lost art” as more sophisticated X-ray and other imaging techniques have been developed. The objective of this report is to challenge clinical orthopaedic surgeons unfamiliar with the technique to explore this simple beside method in the clinical management of fractures. A portable computer-based vibrational analysis device was employed and experiments conducted to objectively evaluate the capabilities of auscultatory percussion techniques. Auscultatory percussion can, with certain limitations, detect the presence of fractures, assess qualitatively the progress of healing, detect delayed or nonunions, and indicate when sufficiently firm continuity has occurred to permit early mobilization or loadbearing. Vibrational assessment is, however, subject to systematic and random errors, and thus cannot always discriminate between the stages of healing in a fractured bone; in addition, various artifacts can lead to significant uncertainty in the diagnosis. Nevertheless, auscultatory percussion is a useful tool in clinical fracture management, and particularly where roentgenographic facilities are inadequate or not available. Computerized vibrational analysis can be used in place of classical percussion/stethoscope methods by those with poor tonal capabilities, or when more objective record keeping is desired.

File P, Wood J P, Kreplick L W. Diagnosis of hip fracture by the auscultatory percussion technique. Am J Emerg Med. 1998 March; 16(2):173-6. a. Traumatic hip pain is a commonly encountered complaint in the emergency department. Occasionally, initial radiographs fail to show a fracture. A delayed diagnosis can result in significant patient morbidity. Diagnostic algorithms have been formulated to evaluate the patient with hip pain and negative initial radiographs. The auscultatory percussion technique can alert the physician of the presence or absence of an occult hip fracture. Consequently, the physician may order a more sophisticated imaging technique.

Tiru M, Goh S H, Low B Y. Use of percussion as a screening tool in the diagnosis of occult hip fractures. Singapore Med J. 2002 September; 43(9):467-9. a. Traumatic hip pain is a common clinical problem in the emergency department. There is significant morbidity in discharging a patient with an undiagnosed undisplaced hip fracture. The auscultatory percussion technique is a useful method to risk stratify patients who present with traumatic hip pain and with normal radiographs. We sought to study the sensitivity and specificity of the auscultatory percussion technique in a prospective study.

Johnston K D, Baker R T, Baker J G. Use of Auscultation and Percussion to Evaluate a Suspected Fracture. JATT Volume 18, Issue 3, May 2013, 18, 1-6

Carter M C. A reliable sign of fractures of the hid or pelvis. N Engl Med. 1981 Nov. 12; 305(20):1220.

A traditional medical technique is to apply a 128 Hz tuning fork to a part of the body and listen with a stethoscope at another part of the body. The medic can determine based on sound whether a fracture is present, sometimes aided by comparison with the symmetrically opposite healthy bone. This technique has been successfully used on a myriad of fracture types, including those that may be difficult or impossible to diagnose using techniques such as physical examination or X-ray. However, this technique relies on the user's training level and judgment for correct diagnosis. Additionally, the potential user population that would benefit from this technique may not commonly carry a tuning fork or even a stethoscope.

There would be value in an alternative diagnosis mechanism that:
i. diagnoses fractures that an X-ray cannot;
ii. is inexpensive;
iii. is intuitive for medically trained or untrained users;
iv. can be used independently of the setting of care, i.e., prehospital, hospital or post-hospital
v. utilizes mostly equipment that is already available to or carried by the user.

This would prevent unnecessary and costly procedures and allow treatment of fractures sooner than the current standard of care.

### SUMMARY OF THE DISCLOSURE

The present invention provides a system that may use a commercially available device that may already be in the possession of the end user, controlled by software, to apply a vibration to a patient and analyze transmission through the patient to predict the probability of pelvic or other bone fractures. A For example, a smartphone or similar device can contain and be controlled by software that controls a vibration generator placed on one or more parts of a person's body and a vibration sensor placed on another. The software can interpret the sensed vibration and determine probability and/or location of the fracture.

A device included in the system may be a smart phone, tablet, laptop computer, or any device capable of loading software and controlling the generation and sensing of vibration, and may include a display device or include a capability to provide a report to a separate display device or to a printer.

Vibration Source:

The vibration source may be reusable or disposable. There is no known optimal frequency setting or range for the vibration to be used. It is possible that different frequencies would be optimal for different geometries or tissue types. It is possible that generating multiple frequencies or a frequency sweep would have a higher predictive value than using one fixed frequency.

Vibration can be generated from a vibrating motor on the device, from the device's speakers, or from a custom external device.

The device may include a factory-installed vibrating motor. A study of common phones (Apple iPhone 2G, Sony U10i, LGKE850, Nokia 6700, and Nokia 3660) showed a vibrator frequency range between 68 Hz and 229 Hz. Other frequencies could be used by other commercial devices.

A cover may be provided for the patient contact portions of the device (similar to probe covers that are used on oral thermometers) to prevent cross contamination and assist with infection control.

The device may include factory-installed speakers. For smartphones, for example, speakers may provide audible sound, generally in the range of 6 Hz to 20,000 Hz.

A cover may be provided for the patient contact portions of the speaker-equipped device (similar to probe covers that are used on oral thermometers) to prevent cross contamination and assist with infection control.

A custom (aftermarket) device may be connected via headphone port, charging port, wireless connection (Bluetooth, ZigBee, Peanut or others) or other means if available. The vibration generator for a custom device may for example be a vibrating motor or speaker.

Custom vibration generators may be able to utilize Ultrasound, i.e., frequencies above 20,000 Hz.

Vibration Detector:

The vibration sensor may be the factory-installed microphone of a device such as a smartphone, for example.

A cover may be provided for the patient contact portions of the vibration sensing device (similar to probe covers that are used on oral thermometers) to prevent cross contamination and assist with infection control.

The vibration sensor may be an aftermarket microphone or other vibration sensor, and may be connected directly to the device via available ports or connected wirelessly via Bluetooth, ZigBee, Peanut, or other connection.

The vibration sensor may be reusable or disposable.

Software or “App”

The system may include software resident in the vibration generator or the vibration sensor device, or both, to prompt the user to input information, such as:
i. location of pain or swelling;
ii. suspected location of injury;
iii. patient identifying and other information, such as age, weight, etc.

The software may prompt the user to place the vibration generator at a location and the vibration detector at another location, after which vibration is initiated.

The software thereafter may prompt the user to place the vibration generator and/or the vibration detector at another (or multiple) locations, based on analysis of the previous vibration event or other factors.

By analysis of the vibration event or events, the software may determine the probability of a fracture, and/or the location of a fracture, and/or the type of fracture present. The analysis may be based on a database of information that may include clinical or cadaveric empirical results, literature, or mathematical modeling.

The software may enable the device to provide a display of the analysis and may provide for transmission of the display and other data on which the analysis has been based, to a storage device or a printer.

The foregoing and other objectives, features, and advantages of the invention will be more readily understood upon consideration of the following detailed description of the invention taken in conjunction with the accompanying drawings.

### BRIEF DESCRIPTION OF THE SEVERAL DRAWINGS

FIG. 1 is a block diagram of a system for acoustic detection of fractures in bones.

FIG. 2 is a simplified pictorial view showing the system in use.

FIGS. 3A and 3B are, taken together, a functional flowchart showing operation of the system.

### DETAILED DESCRIPTION

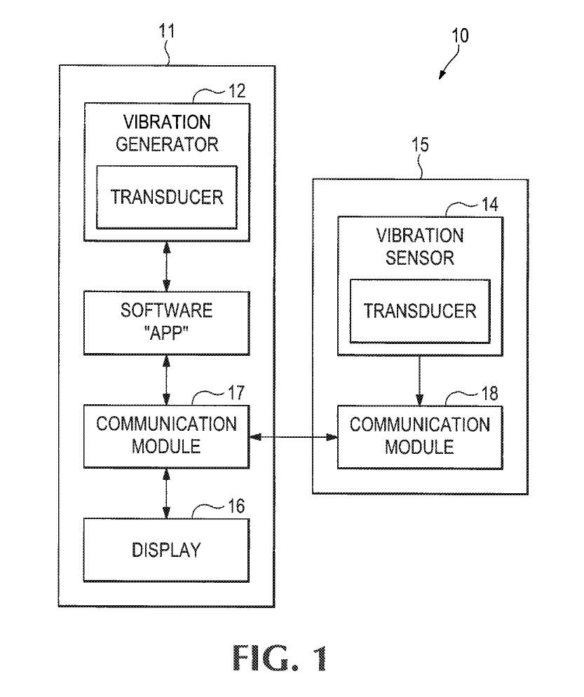

Referring now to the drawings which form a part of the disclosure herein, as shown in FIG. 1, an acoustic bone fracture detection system 10 includes a vibration generator 12, which may be incorporated in a control unit 11, for example, a readily available portable device such as a smart phone. The system 10 also includes a vibration sensor 14, which may similarly be a readily available device 15 such as a smart phone. The system 10 also preferably includes a visual display device 16, which may be a display screen incorporated in the control unit 11 including the vibration generator 12 or the device 15 incorporating the vibration sensor 14, or may incorporate display portions of both of such elements of the system 10. Software and wireless communication elements 17, 18 installed with one or both of the vibration generator 12, and the vibration sensor 14 may be used to communicate between the two elements of the system 10, or the vibration generator 12 may be connected with the vibration sensor 14 by a suitable cable.

Software Decision Tree

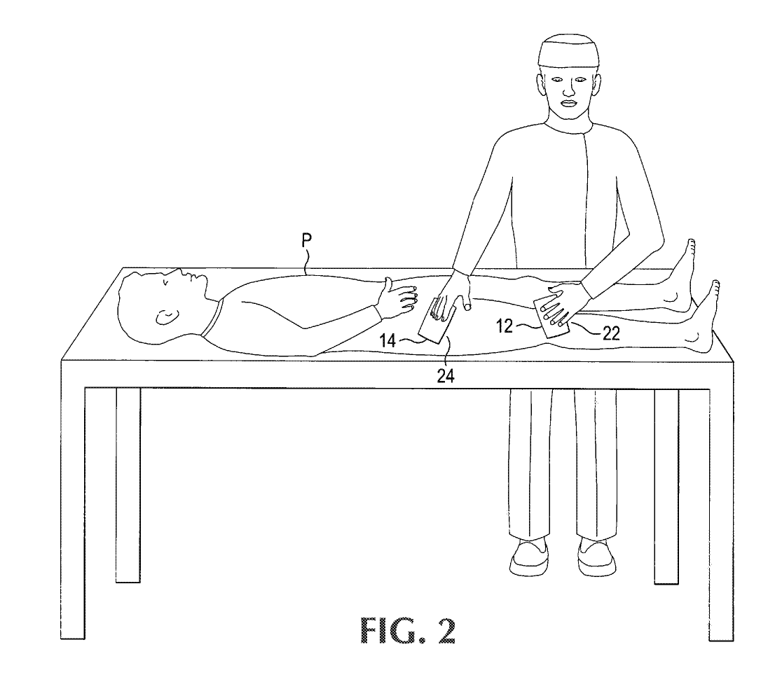

As shown in FIG. 2, the bone fracture detection system 10 is being used on a patient P to 18 in whom a stress fracture of the right femur 20 is suspected.

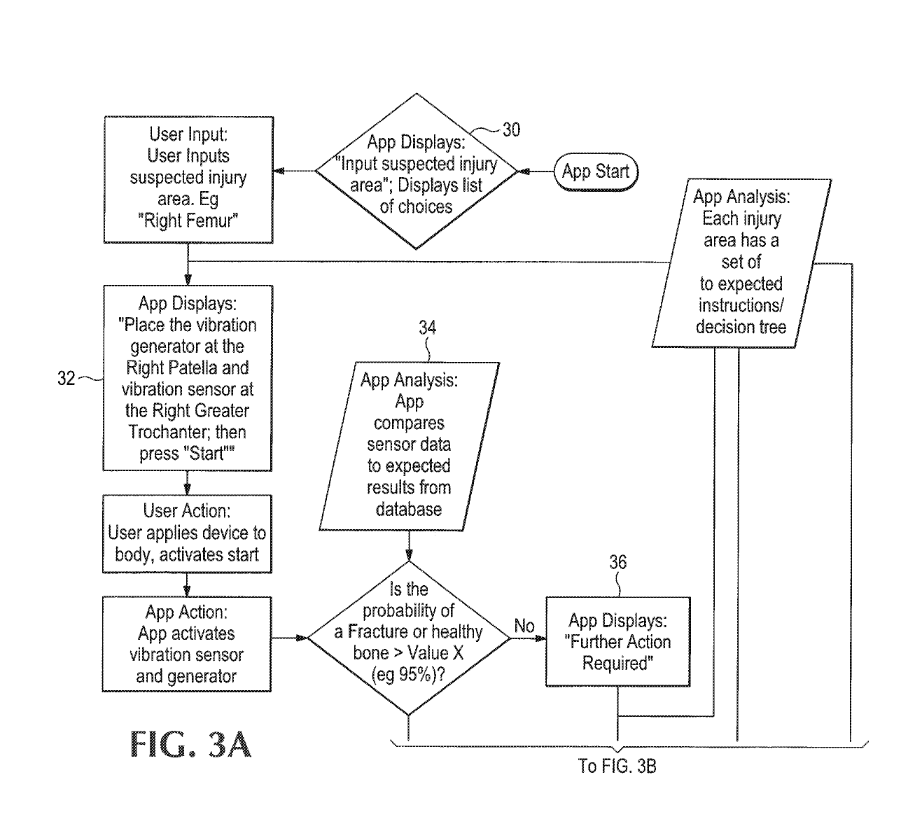

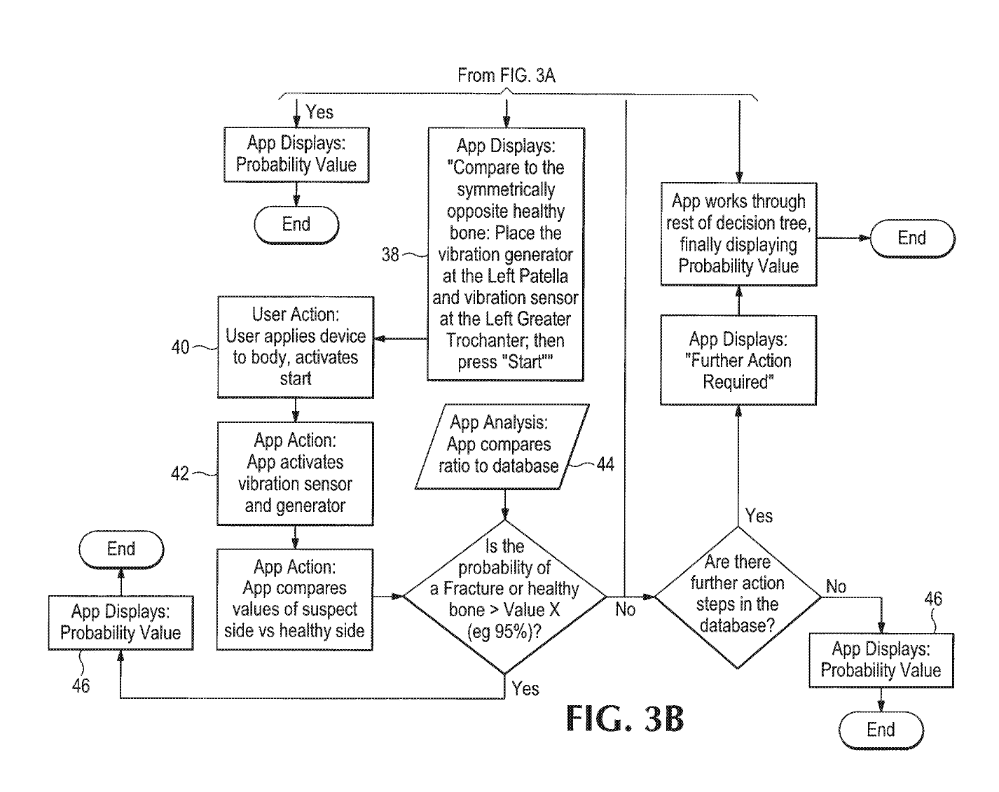

As shown in FIGS. 3A and 3B, the software of the system 10 (hereinafter referred to at times as “the App”) causes instructions to be displayed. A user of the system 10, as prompted by the App, is holding the vibration generator 12 against the right patella 22 of the patient P and the vibration sensor 14 against the patient P at the location of the right greater trochanter 24.

The software App provides instructions and a decision tree to guide a user, such as, for example, the flowchart shown in FIGS. 3A and 3B.

FIGS. 3A and 3B show one possible decision tree, with instruction relating to a suspected right femur stress fracture, for a system 10 in which there is wireless communication between the vibration generator 12 and the vibration sensor 14.

When the App of the system 10 is started, the display 16 shows, for example: “Input suspected injury area”, at 30. The App may display a list of options, an anatomical diagram, and/or a search input bar.

In response, the user inputs 2 suspected injury area via text input, voice input, or selection from a menu of options (text list, anatomical diagram, etc.) In the illustrated example shown in FIG. 3, the user has entered the input “Right femur”.

For each likely injury area of a person, the system software hereinafter, for convenience called the “App,” has a set of instructions, including a decision tree similar to that shown in FIGS. 3A and 3B.

For a suspected right femur stress fracture, the system displays a prompt, as shown in FIG. 3A at 32: “Place the vibration generator 12 (cell phone) at the right patella 22 and the vibration sensor 14 (Bluetooth microphone) at the right greater trochanter 24, then press start (or say start, etc.)”

The User should then apply the vibration generator 12 and vibration sensor 14 to the patient as directed and input a start command on the device.

In response to the “start” command, the App activates vibration sensor 14 and vibration generator 12 with one or more vibrations frequencies according to the App.

Upon sensing vibrations from the patient in response to vibration generated by the device 12, the device provides an analysis as shown at 34 in accordance with the system software.

App Analysis:

Based on expected vs actual results (the analysis may be based on a database of clinical or cadaveric empirical results, on literature, or on mathematical modeling), the App generates a probability value. If the probability of a fracture or healthy bone is greater than a value, for example >95%, the App outputs the result on the display 16. If the probability is less than a value, for example <95%, the App may display a prompt for further action, as at 36.

For example, an App prompt for further action may be:

“Place the vibration generator (cell phone) at the left patella (healthy side) and the vibration sensor (Bluetooth microphone) at the left greater trochanter (healthy side), then press start (or say start, etc.)”, as shown at 38.

In response, a user may take the recommended action:

User applies devices to body, activates start, as at 40.

The control unit 11 will, then, under control of the App, take the following action.

First, the App activates vibration generator 12 and sensor 14 with one or more frequencies as at 42.

Next, the App conducts an analysis as at 44. First, the App compares the values of the suspect side vs the healthy side. Based on the magnitude of difference between the healthy and suspect side, the App generates a probability value. The analysis may be based on a database of clinical or cadaveric empirical results, on literature, or on mathematical modeling. If the probability of a fracture or healthy bone is greater than a number, for example >95%, the App outputs the result on the display 16. If the probability is less than a value, for example <95%, the App may cause the display 16 to show a prompt for further action.

Subsequent Prompts

There may be a series of other further action prompts, which may include positioning the vibration generator 12 and sensor 14 at different anatomical locations, crossing the vibration generator 12 and sensor 14 from the healthy side to the suspect side of the patient P, or moving the vibration generator 12 or sensor 14 along a suspected bone.

When the decision tree is exhausted, the App can display the final probability value as at 46.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A device for diagnosing the probability, location or type of fracture in a bone comprising:
a. a user control unit;
b. a vibration generator;
c. a vibration detector; and
d. a vibration analyzer responsive to the vibration generator and the vibration detector.

2. The device of claim 1, further including a display device arranged to display information provided by the vibration analyzer.

3. The device of claim 1, including a reporting device associated with the vibration analyzer.

4. The device of claim 1 wherein the control unit includes a smartphone.

5. The device of claim 1 wherein the vibration generator is integrated into the control unit.

6. The device of claim 1 wherein the vibration generator is reversibly attached to the control unit.

7. The device of claim 1 wherein the vibration sensor is integrated into the control unit.

8. The device of claim 1 wherein the vibration sensor is reversibly attached to the control unit.

9. The device of claim 1 including a wireless communication unit in the control unit.

10. The device of claim 9 including a wireless communication unit connected with the vibration detector.
